(19) United States
(12) Patent Application Publication
(10) Pub. No.: US 2014/0051976 A1
(43) Pub. Date: Feb. 20, 2014

(54) MRI APPARATUS COMBINED WITH LIGHTFIELD CAMERA

(71) Applicant: ASPECT IMAGING LTD., Shoham (IL)

(72) Inventors: Uri RAPOPORT, Moshav Ben Shemen (IL); Aryeh BATT, Har Hevron (IL)

(21) Appl. No.: 13/943,416

(22) Filed: Jul. 16, 2013

(30) Foreign Application Priority Data
Aug. 15, 2012 (IL) 221491

(51) Int. Cl. G01R 33/48

(52) U.S. Cl. CPC G01R 33/4808
USPC 600/411

(57) ABSTRACT

An MRI apparatus that induces a magnetic resonance signal from an object to be imaged. The apparatus includes: magnet poles for creating a homogeneous magnetic field; and a set of RF coils for generating a radio frequency (RF) excitation pulse in the imaging volume of the apparatus, and for acquiring magnetic resonance signals resulting from the RF excitation pulse. The apparatus also includes a light-field (plenoptic) camera and the object may be imaged concurrently by both MRI and plenoptic channels. The obtained images can be superimposed.

### FIELD OF THE INVENTION

The present invention relates to an MRI apparatus and, more specifically, to MRI apparatus provided with a light-field camera.

### BACKGROUND OF THE INVENTION

Magnetic resonance imaging (MRI) is a medical imaging technique used in radiology to visualize internal structures of the body in detail. MRI makes use of the property of nuclear magnetic resonance (NMR) to image nuclei of atoms inside the body.

An MRI scanner is a device in which the patient lies within a large, powerful magnet where the magnetic field is used to align the magnetization of some atomic nuclei in the body, and radio frequency pulse is used to alter the alignment of this magnetization. This causes the nuclei to precess as the magnetization returns to equilibrium, thereby producing a radio frequency signal related to the local magnetic field experienced by each nucleus. This information is recorded to construct an image of the scanned area of the body. Magnetic field gradients cause nuclei at different locations to precess at different speeds. By using gradients in different directions 2D images or 3D volumes can be obtained in any arbitrary orientation.

JP10165393A discloses an MRI apparatus provided with luminescence means composed of a plurality of illuminants arranged within the MRI apparatus. An MRI image is combined with a luminescence image to improve the diagnostic capabilities of the MRI apparatus.

A light-field camera (plenoptic) camera is a camera that uses a microlens array to capture 4D light field information about a scene. Such light field information can be used to improve the solution of computer graphics and vision-related problems.

U.S. Pat. No. 8,189,065 discloses a method and apparatus for full-resolution light-field capture and rendering. A radiance camera is described in which the microlenses in a microlens array are focused on the image plane of the main lens instead of on the main lens, as in conventional plenoptic cameras. The microlens array may be located at distances from the photosensor greater than the focal length f of the microlenses. Radiance cameras in which the distance of the microlens array from the photosensor is adjustable, and in which other characteristics of the camera are adjustable, are described. Digital and film embodiments of the radiance camera are described. A full-resolution light-field rendering method may be applied to flats captured by a radiance camera to render higher-resolution output images than are possible with conventional plenoptic cameras and rendering methods.

The MRI equipment is provided with means for creating a 3D image of an object of interest and any cross sectional view. In this connection, a combination of an MR image and an optical image looks very promising. Thus, there is a long-felt and unmet need for providing an MRI apparatus which is able to combine an MR image and an optical image such that the aforesaid images are taken concurrently and parallax angles and field depths of the images are identical.

### SUMMARY OF THE INVENTION

It is hence one object of the invention to disclose an MRI apparatus configured for inducing a magnetic resonance signal from an object to be imaged. The aforesaid apparatus comprises a magnet poles for creating a homogeneous magnetic field; and a set of RF coils for generating an RF excitation pulse in the imaging volume of the apparatus, and for acquiring magnetic resonance signals which are due to the RF excitation pulse.

It is a core purpose of the invention to provide the apparatus with a light-field (plenoptic) camera such that the object is imaged concurrently by MRI and plenoptic channels and images obtained from the two channels are superimposed.

Another object of the invention is to disclose the MRI and plenoptic images characterized by an identical parallax angle.

A further object of the invention is to disclose the MRI and plenoptic images characterized by an identical depth of field.

A further object of the invention is to disclose a method of MR imaging by means of inducing a magnetic resonance signal from an object to be imaged. The aforesaid method comprises the steps of (a) providing a MRI apparatus configured for inducing a magnetic resonance signal from an object to be imaged, the apparatus comprising: (i) magnet poles for creating a homogeneous magnetic field; and (ii) a set of RF coils for generating an RF excitation pulse in the imaging volume of the apparatus, and for acquiring magnetic resonance signals which are due to the RF excitation pulse; (b) concurrently imaging the object; (c) rendering MR and plenoptic images; and (d) superimposing the MR and plenoptic images.

It is a further object of the present invention to disclose an MRI apparatus for providing superimposed MRI and plenoptic images, wherein said apparatus comprises: magnet poles for creating a homogeneous magnetic field; a set of RF coils for generating an RF excitation pulse in the imaging volume of the apparatus, and for acquiring magnetic resonance signals which are due to the RF excitation pulse; MR imaging means for converting said magnetic resonance signals into a magnetic resonance image; a light-field (plenoptic) camera configured to obtain plenoptic images concurrently with said magnetic resonance images; and, superposition means for superimposing said magnetic resonance and plenoptic images.

It is a further object of the present invention to disclose the MRI apparatus as defined in any of the above, wherein said magnetic resonance images and said plenoptic images are characterized by an identical parallax angle.

It is a further object of the present invention to disclose the MRI apparatus as defined in any of the above, wherein said magnetic resonance images and said plenoptic images are characterized by an identical depth of field.

It is a further object of the present invention to disclose the MRI apparatus as defined in any of the above, wherein said magnet poles are constructed from permanent magnets.

It is a further object of the present invention to disclose a method for providing superimposed magnetic resonance and plenoptic images, comprising: providing an MRI apparatus as defined in any of the above; placing an object within a field of view of said MRI apparatus; using said MR imaging means to produce a magnetic resonance image characterized by a parallax angle and a depth of field; using said plenoptic camera to produce, concurrently with said magnetic resonance image, a plenoptic image characterized by a parallax angle and a depth of field; rendering magnetic resonance and plenoptic images; and, superimposing said magnetic resonance and plenoptic images.

It is a further object of the present invention to disclose such a method, wherein said step of producing a plenoptic image comprises producing a plenoptic image having a parallax angle identical to said parallax angle of said magnetic resonance image.

It is a further object of the present invention to disclose the method as defined in any of the above, wherein said step of producing a plenoptic image comprises producing a plenoptic image having a depth of field identical to said depth of field of said magnetic resonance image.

It is a further object of the present invention to disclose the method as defined in any of the above, wherein said step of providing an MRI apparatus comprises providing an MRI apparatus comprising pole pieces constructed from permanent magnets.

### BRIEF DESCRIPTION OF THE DRAWINGS

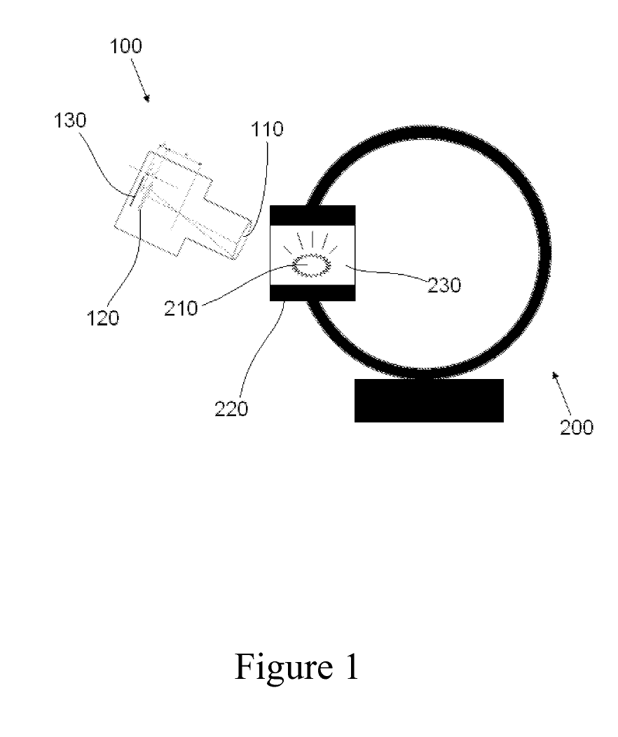

In order to understand the invention and to see how it may be implemented in practice, a plurality of embodiments is adapted to now be described, by way of non-limiting example only, with reference to the accompanying drawing, in which FIG. 1 is a schematic view of an MRI apparatus a light-field (plenoptic) camera.

### DETAILED DESCRIPTION OF THE INVENTION

The following description is provided so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide an MRI apparatus configured for inducing a magnetic resonance signal from an object to be imaged and a method of using the same.

Reference is now made to FIG. 1, presenting a schematic view of an MRI apparatus of the present invention. The magnetic portion 200 is configured for inducing a magnetic resonance signal from an object to be imaged. Magnet poles 220 create a homogeneous magnetic field. A set of RF coils (not shown) generates an RF excitation pulse in the imaging volume 230 of the apparatus and acquires magnetic resonance signals from a sample 210 to be examined which are due to the RF excitation pulse. A plenoptic camera 100 is oriented for receiving optical radiation (i.e. radiation of wavelength shorter than microwave) emitted or reflected or scattered by the sample 210. Detection of bioluminescent or fluorescent radiation is in the scope of the present invention.

The plenoptic camera 100 comprises a main lens 110 and a microlens array 120. The aforesaid array 120 focuses a matrix of micro images on an image detector (CCD) 130.

Software known in the art is able to refocus an image obtained by the plenoptic camera and to change a parallax angle (point of view). This feature of the proposed technical solution provides an opportunity of fine adjustment of MR and plenoptically rendered images. The images examined in association with each other improve diagnostic capabilities.

In one embodiment of the present invention, it comprises an MRI apparatus configured for inducing a magnetic resonance signal from an object to be imaged is disclosed. The aforesaid apparatus comprises magnet poles for creating a homogeneous magnetic field and a set of RF coils for generating an RF excitation pulse in the imaging volume of the apparatus and for acquiring magnetic resonance signals which are due to the RF excitation pulse. In some embodiments of the invention, the magnet poles are those of a permanent magnet or an electromagnet.

It is a core feature of the present invention to provide the apparatus with a light-field (plenoptic) camera such that said object is imaged concurrently by MRI and plenoptic channels and the MRI and plenoptic images are superimposed. In preferred embodiments, the apparatus is provided with software known in the art that performs the superposition of the two images.

In some embodiments of the apparatus, the MRI and plenoptic images are characterized by an identical parallax angle.

In some embodiments of the apparatus, the MRI and plenoptic images are characterized by an identical depth of field. The depth of field can be chosen manually by the operator or set automatically by the apparatus.

In accordance with another embodiment of the present invention, a method of MR imaging by means of inducing a magnetic resonance signal from an object to be imaged is disclosed. The aforesaid method comprises the steps of (a) providing a MRI apparatus configured for inducing a magnetic resonance signal from an object to be imaged, said apparatus comprising: (i) a magnet poles for creating a homogeneous magnetic field; and (ii) a set of RF coils for generating an RF excitation pulse in the imaging volume of the apparatus and for acquiring magnetic resonance signals which are due to the RF excitation pulse; (b) concurrently imaging said object; (c) rendering MR and plenoptic images; and (d) superimposing said MR and plenoptic images. The MR imaging can be performed by using any type of MR imaging instrument known in the art. In particular, MR imaging instruments in which the magnetic field is produced by a permanent magnet are considered by the inventor to be within the scope of the invention.

1-6. (canceled)

7. An MRI apparatus for providing superimposed MRI and plenoptic images, wherein said apparatus comprises:
magnet poles for creating a homogeneous magnetic field;
a set of RF coils for generating an RF excitation pulse in the imaging volume of the apparatus, and for acquiring magnetic resonance signals which are due to the RF excitation pulse;
MR imaging means for converting said magnetic resonance signals into a magnetic resonance image;
a light-field (plenoptic) camera configured to obtain plenoptic images concurrently with said magnetic resonance images; and,
superposition means for superimposing said magnetic resonance and plenoptic images.

8. The MRI apparatus according to claim 7, wherein said magnetic resonance images and said plenoptic images are characterized by an identical parallax angle.

9. The MRI apparatus according to claim 7, wherein said magnetic resonance images and said plenoptic images are characterized by an identical depth of field.

10. The MRI apparatus according to claim 7, wherein said magnet poles are constructed from permanent magnets.

11. A method for providing superimposed magnetic resonance and plenoptic images, comprising:
providing an MRI apparatus according to claim 7,
placing an object within a field of view of said MRI apparatus;
using said MR imaging means to produce a magnetic resonance image characterized by a parallax angle and a depth of field;
using said plenoptic camera to produce, concurrently with said magnetic resonance image, a plenoptic image characterized by a parallax angle and a depth of field;
rendering magnetic resonance and plenoptic images; and,
superimposing said magnetic resonance and plenoptic images.

12. The method according to claim 11, wherein said step of producing a plenoptic image comprises producing a plenoptic image having a parallax angle identical to said parallax angle of said magnetic resonance image.

13. The method according to claim 11, wherein said step of producing a plenoptic image comprises producing a plenoptic image having a depth of field identical to said depth of field of said magnetic resonance image.

14. The method according to claim 11, wherein said step of providing an MRI apparatus comprises providing an MRI apparatus comprising pole pieces constructed from permanent magnets.
